(19) United States
(12) Patent Application Publication
(10) Pub. No.: US 2026/0306686 A1
(43) Pub. Date: Oct. 8, 2026

(54) CLEANER ASSEMBLY WITH ATTACHABLE OZONE DISPENSER

(71) Applicant: O3WATERWORKS, LLC, Miami, FL (US)

(72) Inventor: Jonathan Nussbaum, Miami, FL (US)

(21) Appl. No.: 19/563,359

(22) Filed: Mar. 11, 2026

Related U.S. Application Data
(60) Provisional application No. 63/784,748, filed on Apr. 7, 2025.

(51) Int. Cl. A61L 2/183; A47L 7/00; A47L 13/16; A47L 13/22; A47L 13/254; A47L 13/42; A61L 103/75

(52) U.S. Cl. CPC A61L 2/183; A47L 7/0061; A47L 13/16; A47L 13/22; A47L 13/254; A47L 13/42; A61L 2103/75; A61L 2202/11; A61L 2202/15; A61L 2202/16; A61L 2202/17

(57) ABSTRACT

A cleaner assembly (10) for cleaning a floor (32) includes a cleaning device (12), and an ozone dispenser (14) that is attachable to the cleaning device (12). The ozone dispenser (14) can be fixedly attached to the cleaning device (12), or the ozone dispenser (14) can be removably attachable to the cleaning device (12), and can thus be used as a stand-alone cleaning mechanism. The ozone dispenser (14) includes a water reservoir (240) that is configured to receive and retain a volume of source water (16) from a water source (18); and an ozone generator (242) that is positioned one of substantially within and substantially adjacent to the water reservoir (240), the ozone generator (242) being configured to generate ozone within the source water (16) retained within the water reservoir (240) to generate ozonated water (20).

### RELATED APPLICATION

This application claims priority on U.S. Provisional Application Ser. No. 63/784,748, filed on Apr. 7, 2025, and entitled “MOP WITH OZONE DISPENSER ATTACHED”. As far as permitted, the contents of U.S. Provisional Application Ser. No. 63/784,748 are incorporated in their entirety herein by reference.

### BACKGROUND

For many years, ozone, as a known powerful oxidizer, has been effectively used as a disinfecting agent in a wide variety of industries worldwide to control odors and kill germs, viruses, bacteria, molds and yeasts, including in wastewater plants, water parks, zoos, aquariums, food manufacturing, and water bottling.

It is desired to further expand potential uses for ozone, and to make ozone as a cleaner and disinfectant more accessible to the residential user.

### SUMMARY

The present invention is directed toward a cleaner assembly for cleaning a surface. In various embodiments, the cleaner assembly includes a cleaning device, and an ozone dispenser that is attachable to the cleaning device.

In some embodiments, the ozone dispenser is removably attachable to the cleaning device.

In certain embodiments, the ozone dispenser is fixedly attached to the cleaning device.

In various embodiments, the cleaning device includes (i) a device body having a first body end and an opposed second body end, (ii) a device head that is coupled to the first body end, and (iii) a device handle that is coupled to the second body end.

In some embodiments, the device head is movably coupled to the first body end of the device body.

In certain embodiments, the device head includes a substantially planar floor-facing surface that is configured to face the floor during a cleaning procedure.

In various embodiments, the cleaner assembly further includes at least one cleaning pad that is coupled to the floor-facing surface. The at least one cleaning pad can be removably coupled to the floor-facing surface.

In certain embodiments, the cleaning device further includes a dispenser receiver to is configured to receive and retain the ozone dispenser.

In some embodiments, the cleaning device further includes a dispenser activator that is coupled to the device handle, the dispenser activator being usable to selectively activate the ozone dispenser.

In various embodiments, the dispenser activator includes an electronic mechanism that is electronically linked to the ozone dispenser to selectively activate the ozone dispenser.

In certain embodiments, the dispenser activator includes a manual mechanism that is mechanically linked to the ozone dispenser to selectively activate the ozone dispenser.

In some embodiments, the ozone dispenser includes a water reservoir that is configured to receive and retain a volume of source water from a water source; and an ozone generator that is positioned one of substantially within and substantially adjacent to the water reservoir, the ozone generator being configured to generate ozone within the source water retained within the water reservoir to generate ozonated water.

In various embodiments, the ozone generator is an electrolytic cell-type ozone generator that includes an electrolytic cell that is positioned within the dispenser reservoir.

In certain embodiments, the ozone generator is a corona discharge-type ozone generator.

In some embodiments, the ozone dispenser further includes a spray activator that is configured to selectively activate the ozone dispenser when the ozone dispenser is not attached to the cleaning device.

In certain embodiments, the cleaning device is a mop.

In various embodiments, the cleaning device is a vacuum cleaner.

The present invention is further directed toward a cleaner assembly for cleaning a surface, the cleaner assembly including (A) a cleaning device including (i) a device body having a first body end and an opposed second body end; (ii) a device head that is movably coupled to the first body end, the device head includes a substantially planar floor-facing surface that is configured to face the floor during a cleaning procedure; (iii) a device handle that is coupled to the second body end; and (iv) a dispenser activator that is configured to selectively activate the ozone dispenser, the dispenser activator including one of an electronic mechanism that is electronically linked to the ozone dispenser to selectively activate the ozone dispenser and a manual mechanism that is mechanically linked to the ozone dispenser to selectively activate the ozone dispenser; and (B) an ozone dispenser that is attachable to the cleaning device, the ozone dispenser including (i) a water reservoir that is configured to receive and retain a volume of source water from a water source; (ii) an ozone generator that is positioned one of substantially within and substantially adjacent to the water reservoir, the ozone generator being configured to generate ozone within the source water retained within the water reservoir to generate ozonated water; (iii) a power source that is configured to provide power to the ozone generator; and (iv) a dispenser nozzle through which the ozonated water is dispensed from within the water reservoir.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1A is a simplified schematic perspective view illustration of an embodiment of a cleaner assembly having features of the present invention, the cleaner assembly including a cleaning device and an embodiment of an ozone generating and dispensing device that is attached to the cleaning device;

FIG. 1B is another simplified schematic perspective view illustration of the cleaner assembly illustrated in FIG. 1A, the ozone generating and dispensing device having been removed and/or detached from the cleaning device;

FIG. 2A is a simplified schematic front view illustration of an embodiment of the ozone generating and dispensing device that can be used as part of the cleaner assembly illustrated in FIG. 1A;

FIG. 2B is a simplified schematic side view illustration of the ozone generating and dispensing device illustrated in FIG. 2A;

FIG. 2C is a simplified schematic top view illustration of the ozone generating and dispensing device illustrated in FIG. 2A;

FIG. 3 is a simplified schematic perspective view illustration of another embodiment of the cleaner assembly; and

FIG. 4 is a simplified schematic perspective view illustration of still another embodiment of the cleaner assembly.

While embodiments of the present invention are susceptible to various modifications and alternative forms, specifics thereof have been shown by way of example and drawings, and are described in detail herein. It is understood, however, that the scope herein is not limited to the particular embodiments described. On the contrary, the intention is to cover modifications, equivalents, and alternatives falling within the spirit and scope herein.

### DESCRIPTION

Embodiments of the present invention are described herein in the context of a cleaner assembly that is configured to provide enhanced cleaning capabilities to a typical residential user. In particular, in various embodiments, the cleaner assembly includes a cleaning device (such as a mop or a vacuum cleaner, in certain non-exclusive embodiments), and an ozone generating and dispensing device (also referred to herein as an “ozone dispenser”) that is attachable to the cleaning device. In alternative embodiments, the ozone dispenser can be fixedly attached to the cleaning device or removably attached to the cleaning device. In embodiments in which the ozone dispenser is removably attached to the cleaning device, the ozone dispenser can be used as a stand-alone cleaning agent or cleaning mechanism, independent of its use as part of the cleaner assembly.

Those of ordinary skill in the art will realize that the following detailed description of the present invention is illustrative only and is not intended to be in any way limiting. Other embodiments of the present invention will readily suggest themselves to such skilled persons having the benefit of this disclosure. Reference will now be made in detail to implementations of the present invention as illustrated in the accompanying drawings. The same or similar nomenclature and/or reference indicators will be used throughout the drawings and the following detailed description to refer to the same or like parts.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application-related and business-related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

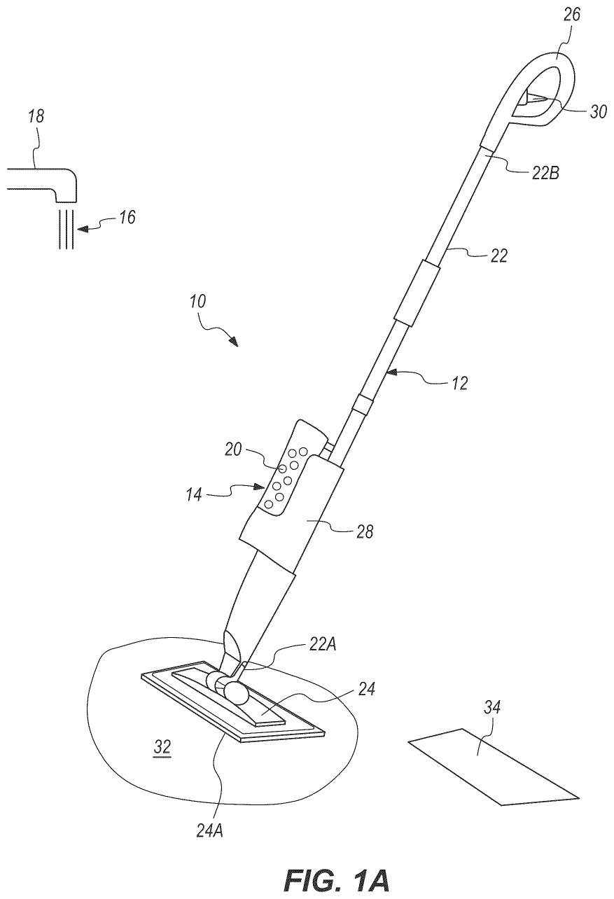

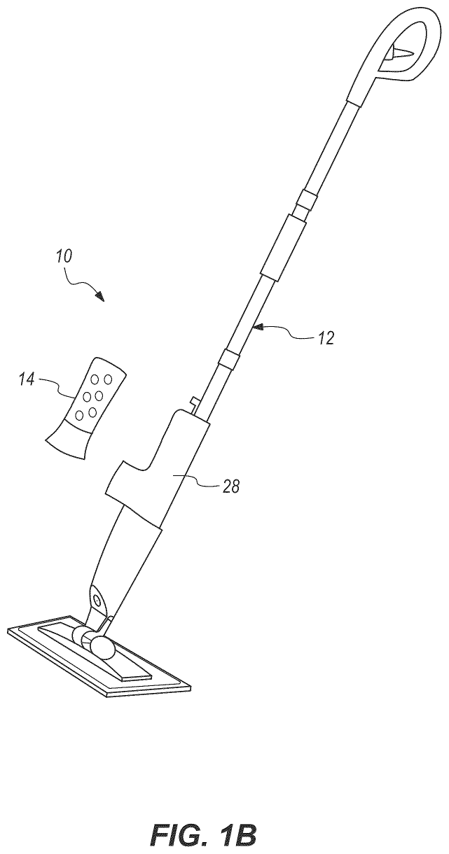

FIG. 1A is a simplified schematic perspective view illustration of an embodiment of a cleaner assembly 10 having features of the present invention. The design of the cleaner assembly 10 can be varied. In various embodiments, as shown, the cleaner assembly 10 includes a cleaning device 12, and an embodiment of an ozone generating and dispensing device 14 (also referred to herein as an “ozone dispenser”) that is attachable to the cleaning device 12. Alternatively, the cleaner assembly 10 can have more components than what is illustrated and described herein, and/or each of the components of the cleaner assembly 10 can have a somewhat different design and function than what is described in detail herein.

The cleaner assembly 10 can include any suitable type of cleaning device 12. For example, in certain embodiments, such as shown in FIG. 1A, the cleaning device 12 can be a mop. Alternatively, in other embodiments, the cleaning device 12 can be a vacuum cleaner, such as a wet-dry vacuum cleaner. Still alternatively, in yet other embodiments, the cleaning device 12 can be another suitable type of cleaning device.

As an overview, in various embodiments, the ozone dispenser 14 is configured to receive a volume of source water 16 from a water source 18, such as ordinary tap water from an ordinary tap, in order to generate a corresponding volume of ozonated water 20 (illustrated as a plurality of small circles), and to then selectively dispense the ozonated water 20 as a cleaning agent. As attached to the cleaning device 12, the ozone dispenser 14 can be utilized to selectively dispense the ozonated water 20 as a cleaning agent to be used specifically in conjunction with the cleaning device 12.

In certain embodiments, such as the embodiment shown in FIG. 1A, the ozone dispenser 14 can be removably attached to the cleaning device 12. It is appreciated that in embodiments in which the ozone dispenser 14 is removably attached to the cleaning device 12, (i) the ozonated water 20 from the ozone dispenser 14 can be used as a cleaning agent in conjunction with use of the cleaning device 12 as part of the cleaner assembly 10, and/or (ii) the ozonated water 20 from the ozone dispenser 14 can be used as a cleaning agent or cleaning mechanism independently of its use as part of the cleaner assembly 10.

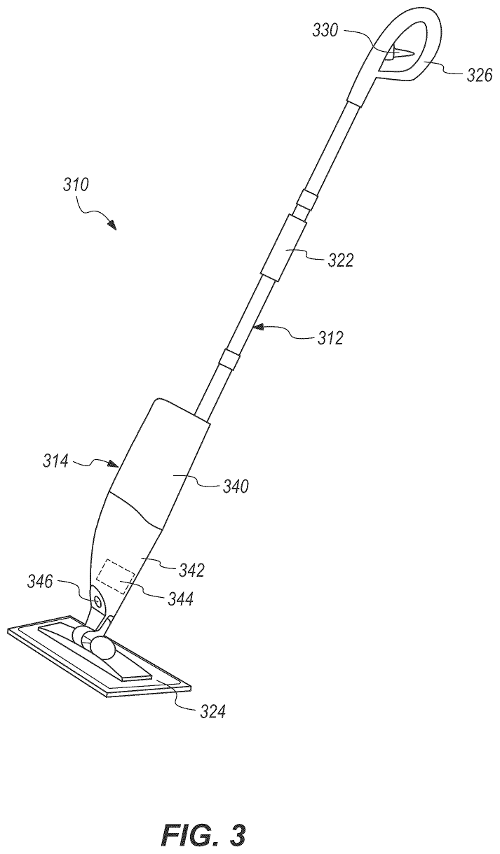

In other embodiments, the ozone dispenser 14 can be fixedly attached to the cleaning device 12. In such embodiments, the ozonated water 20 from the ozone dispenser 14 is usable as a cleaning agent in conjunction with use of the cleaning device 12 as part of the cleaner assembly 10. An example of one such embodiment is illustrated and described in detail herein below in relation to FIG. 3.

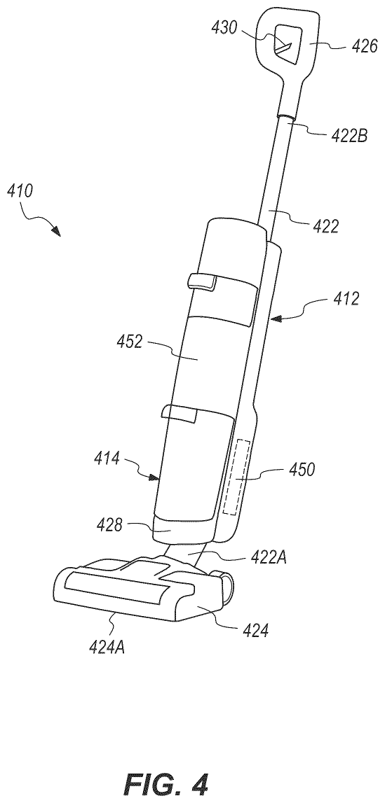

The cleaning device 12 can have any suitable design. As illustrated in the embodiment shown in FIG. 1A, the cleaning device 12 includes a device body 22, a device head 24, a device handle 26, a dispenser receiver 28, and a dispenser activator 30. Alternatively, the cleaning device 12 can have more components or fewer components than what is specifically shown in FIG. 1A. For example, in embodiments in which the cleaning device 12 is a vacuum, the cleaning device 12 can further include a suction assembly 450 (illustrated in phantom in FIG. 4) and a collection receptacle 452 (illustrated in FIG. 4).

In many embodiments, the device body 22 is generally cylindrical pole-shaped, and extends between the device head 24 and the device handle 26. More particularly, as shown, the device body 22 includes a first body end 22A that is coupled to the device head 24, and a second body end 22B that is coupled to the device handle 26. As such, the device body 22 comprises a majority of a length of the cleaning device 12. It is appreciated that the device body 22 can have any suitable length, with different lengths for the device body 22 being made available for users of different heights. In certain embodiments, the device body 22 can be selectively extendable, such as through the use of telescopingly connected body sections, in order that the device body 22 can be of alternative suitable lengths.

The device body 22 can be made from any suitable materials. For example, in certain non-exclusive alternative embodiments, the device body 22 can be made from wood, plastic, fiberglass, aluminum, or steel. Alternatively, the device body 22 can be made from other suitable materials.

The device head 24 can be coupled to the first body end 22A of the device body 22 in any suitable manner. In certain embodiments, the device head 24 can be movably coupled to the first body end 22A of the device body 22. For example, in some embodiments, the device head 24 can be pivotably and/or rotatably coupled to the first body end 22A of the device body 22 to enable the device head 24 to pivot and/or rotate relative to the device body 22.

The device head 24 is configured to engage a floor 32 or other designated surface(s), during use of the cleaner assembly 10. As used herein, the term “floor” or other designated surface(s) can also be referred to simply as a “surface”. Also, it is recognized that the term “floor” and/or “surface” can include other level and/or non-level surfaces, such as walls, ceilings, stairways, ramps, fireplace hearths, countertops, patios, walkways, hallways, driveways, etc., as non-exclusive alternative examples. The design of the device head 24 can be varied. In many embodiments, as shown in FIG. 1A, the device head 24 can be substantially rectangular-shaped and can include a substantially planar floor-facing surface 24A that is configured to face in a generally downward direction, such as toward the surface 32, during use of the cleaner assembly 10. It is appreciated that in embodiments in which the device head 24 is movably coupled to the first body end 22A of the device body 22, it is much easier for the user to maintain the floor-facing surface 24A substantially fully adjacent to the surface 32 during use of the cleaner assembly 10, even as the orientation of the device body 22 relative to the surface 32 changes. It is further appreciated that in such embodiments in which the device head 24 is movably coupled to the first body end 22A of the device body 22, the generally floor-facing surface 24A can be manipulated to face a different designated surface, such as along base boards that are substantially adjacent to the surface 32. Alternatively, the device head 24 can have another suitable design.

In certain embodiments, the device head 24 can be configured such that cleaning elements 34 can be coupled to the floor-facing surface 24A. In some embodiments, the cleaning elements 34 can be removably coupled to the floor-facing surface 24A of the device head 24 With this design, the cleaning elements 34 can be quickly and easily removed and disposed of, and then replaced after use, so that a clean, cleaning element 34 can be coupled to the floor-facing surface 24A for subsequent cleaning procedures. It is appreciated that a single cleaning element 34 is illustrated in FIG. 1A spaced apart from the floor-facing surface 24A, as the cleaning element 34 would not be clearly visible in FIG. 1A when coupled to the floor-facing surface 24A of the device head 24.

The device head 24 can be made from any suitable materials. For example, in certain non-exclusive alternative embodiments, the device head 24 can be made from wood, plastic, fiberglass, aluminum, or steel. Alternatively, the device head 24 can be made from other suitable materials.

The device handle 26 can be coupled to the second body end 22B of the device body 22 in any suitable manner. In certain embodiments, the device handle 26 can be fixedly coupled to the second body end 22B of the device body 22, such as with an adhesive, with one or more screws, or in another suitable manner.

The device handle 26 is intended to be grasped and held by the user during use of the cleaner assembly 10. It is appreciated that the device handle 26 can have any suitable design for purposes of enabling the user to comfortably grasp and hold the device handle 26 during use of the device assembly 10. For example, in certain embodiments, the device handle 26 can have an ergonomic design that enables improved comfort for the user of the cleaning device 12 and/or the cleaner assembly 10. In some embodiments, the device handle 26 can be padded to further assist in the comfort for the user.

It is further appreciated that, in some embodiments, as described in greater detail herein below, the device handle 26 can include features the enable the user to activate the ozone dispenser 14 without letting go of the device handle 26.

The device handle 26 can be made from any suitable materials. For example, in certain non-exclusive alternative embodiments, the device handle 26 can be made from wood, plastic, fiberglass, aluminum, or steel. Alternatively, the device handle 26 can be made from other suitable materials.

As illustrated, the dispenser receiver 28 is provided along the length of the device body 22 and is configured to receive and retain the ozone dispenser 14 as the ozone dispenser 14 is attached to the cleaning device 12. It is appreciated that the dispenser receiver 28 can be positioned at any suitable position along the length of the device body 22. However, it is further appreciated that in various embodiments it is preferred that the dispenser receiver 28 be positioned near the first body end 22A of the device body 22, and/or relatively close to the device head 24. With the dispenser receiver 28 being positioned relatively close to the device head 24, when the ozone dispenser 14 is received and retained within the dispenser receiver 28, dispensing of the ozonated water 20 during use of the cleaner assembly 10 can occur relatively close to the surface 32. With such design, the ozonated water 20 can be sprayed along the surface 32 relatively close to the device head 24 so that cleaning can occur more quickly and efficiently than if the ozonated water 20 is sprayed farther away from the device head 24. Moreover, the shorter distance the ozonated water 20 has to travel to reach the surface 32, the more precise and/or accurate the spraying of the ozonated water 20 from the ozone dispenser 14 can be.

In some embodiments, such as the embodiment illustrated in FIGS. 1A and 1B, the dispenser receiver 28 can be configured to removably receive and retain the ozone dispenser 14. Alternatively, in other embodiments, the dispenser receiver 28 can be configured to fixedly receive and retain the ozone dispenser 14. As provided herein, in embodiments in which the dispenser receiver 28 removably receives and retains the ozone dispenser 14, the ozone dispenser 14 can be removed from the dispenser receiver 28 for potential use as a stand-alone cleaning mechanism or cleaning agent.

The dispenser activator 30 is configured to selectively activate the ozone dispenser 14 so that the ozonated water 20 is sprayed onto the surface 32 near the device head 24. In many embodiments, such as shown in FIG. 1A, the dispenser activator 30 can be coupled to the device handle 26. Alternatively, the dispenser activator 30 can be positioned in another suitable manner, such as at any suitable position along a length of the device body 22.

The dispenser activator 30 can have any suitable design for effectively activating the ozone dispenser 14 to spray the ozonated water 20 onto the surface 32 near the device head 24. For example, in one non-exclusive embodiment, the dispenser activator 30 can include an electronic trigger (or other electronic mechanism) that is electronically linked to the ozone dispenser 14, such that activating the electronic trigger sends an electronic signal to the ozone dispenser 14 so that the ozonated water 20 is sprayed onto the surface 32 near the device head 24. In one embodiment, the electronic signal can be sent wirelessly, such as via a Bluetooth-type attachment. Alternatively, in another non-exclusive embodiment, the dispenser activator 30 can have a manual trigger design, such that pulling back on the manual trigger causes the ozone dispenser 14 to spray the ozonated water 20 onto the surface 32 near the device head 24. In such embodiment, an appropriate mechanical linkage assembly, of any suitable design, is provided between the dispenser activator 30 and the ozone dispenser 14 such that manipulation of the dispenser activator 30 causes the desired activation of the ozone dispenser 14. Still alternatively, in another embodiment, the dispenser activator 30 can have a push-button design, which can be part of either an electronic or manual dispenser activator, such that pushing the button causes the ozone dispenser 14 to spray the ozonated water 20 onto the surface 32 near the device head 24. Yet alternatively, the dispenser activator 30 can have another suitable design.

In one embodiment, when the ozone dispenser 14 is effectively activated through manipulation of the dispenser activator 30, the ozone dispenser 14 can effectively spray or dispense the ozonated water 20 onto the surface 32 through the ozone dispenser 14 itself. Stated in another manner, in such embodiment, the ozonated water 20 can be sprayed directed from the ozone dispenser 14 onto the surface 32. Alternatively, in another embodiment, when the ozone dispenser 14 is effectively activated through manipulation of the dispenser activator 30, the ozone dispenser 14 and/or the cleaner assembly 10 as a whole, can be configured to effectively spray or dispense the ozonated water 20 from the ozone dispenser 14, such as into and through a dispenser coordinator (not shown) and/or the device head 24, and onto the surface 32. With such design, the ozonated water 20 will only need to travel a short distance from the cleaning device 12 before contacting the surface 32. In such alternative embodiment, it is appreciated that a fluid path from the ozone dispenser 14 into and through the dispenser coordinator and/or the device head 24 can be created in any suitable manner.

The ozone dispenser 14 can have any suitable design for purposes of effectively receiving a volume of source water 16 from the water source 18, generating ozone within the source water 16 in order to generate the corresponding volume of ozonated water 20, and then selectively dispensing the ozonated water 20 as a cleaning agent. Certain non-exclusive embodiments for the ozone dispenser 14 will be illustrated and described herein below in relation to subsequent Figures.

In certain embodiments, the ozone dispenser 14 can be positioned within the dispenser receiver 28 in a generally upright manner, such as in the orientation in which the ozone dispenser 14 would be typically utilized when being used as a stand-alone cleaning mechanism. Alternatively, in other embodiments, the ozone dispenser 14 can be positioned within the dispenser receiver in an inverted manner, such as in an orientation in which the ozone dispenser 14 is generally upside down relative to how the ozone dispenser 14 would typically be utilized when being used as a stand-alone cleaning mechanism.

FIG. 1B is another simplified schematic perspective view illustration of the cleaner assembly 10 illustrated in FIG. 1A, the ozone dispenser 14 having been removed from the cleaning device 12. More specifically, FIG. 1B illustrates the ozone dispenser 14 as one that is removably attached to the cleaning device 12. As illustrated, the ozone dispenser 14 having been removed from the cleaning device 12 is no longer being received and retained within the dispenser receiver 28 of the cleaning device 12. Thus, at this point, the ozone dispenser 14 can be usable in a stand-alone manner for performing cleaning procedures, independent of its use as a cleaning mechanism or cleaning agent as part of the cleaner assembly 10.

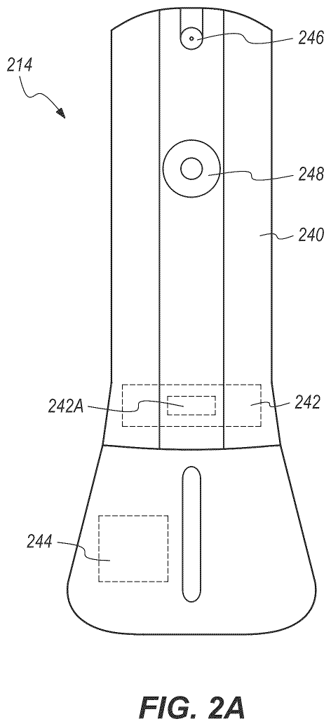

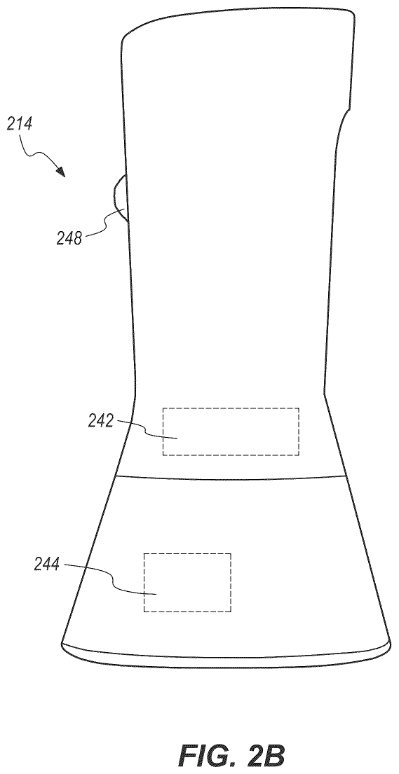

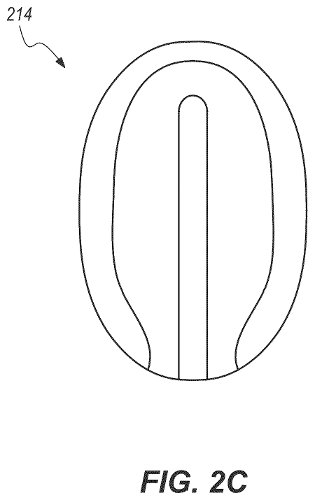

FIG. 2A is a simplified schematic front view illustration of an embodiment of the ozone dispenser 214 that can be used as part of the cleaner assembly 10 illustrated in FIG. 1A. While the embodiment of the ozone dispenser 214 illustrated and described herein is usable as a stand-alone device, and thus could be part of an embodiment of the cleaner assembly 10 in which the ozone dispenser 214 is removably attached to the cleaning device 12 (illustrated in FIG. 1A), certain modifications will also be noted herein for how the ozone dispenser 214 could be more usable as part of an embodiment of the cleaner assembly 10 in which the ozone dispenser is fixedly attached to the cleaning device 12.

The design of the ozone dispenser 214 can be varied. In various embodiments, the ozone dispenser 214 can include a water reservoir 240, an ozone generator 242 (illustrated in phantom), a power source 244 (illustrated in phantom), a dispenser nozzle 246, and a spray activator 248. Alternatively, the ozone dispenser 214 can include more components or fewer components than those specifically illustrated and described in relation to FIG. 2A.

The water reservoir 240 is configured to selectively receive and retain a volume of source water 16 (illustrated in FIG. 1A) from a water source 18 (illustrated in FIG. 1A), such as ordinary tap water from a water tap. In many embodiments, the water reservoir 240 can include a water receiving aperture (not shown), through which the source water 16 can be received into the water reservoir 240. In such embodiments, the water receiving aperture would typically be closeable and/or sealable such that once the source water 16 is received into the water reservoir 240, the water receiving aperture would be closed and/or sealed so that the source water 16 would not leak out of the water reservoir 240 in an undesired manner.

The water reservoir 240 can have any suitable size and shape for purposes of effectively receiving and retaining the source water 16 from the water source 18. Additionally, the water reservoir 240 can be formed from any suitable materials.

It is appreciated that in embodiments in which the ozone dispenser 214 is fixedly attached to the cleaning device 12, the water reservoir 240 could be permanently, fixedly attached and/or built-in to the dispenser receiver 28 (illustrated in FIG. 1A) of the cleaning device 12, or the ozone dispenser 214 (including the water reservoir 240 and other components incorporated therein) could be fixedly secured to the device body 22 (illustrated in FIG. 1A) without the need for a specific dispenser receiver 28. Thus, in such embodiment, the water reservoir 240 can be considered to be part of the cleaning device 12 itself.

The ozone generator 242 is configured to generate ozone in the source water 16 in order to convert the source water 16 to the desired ozonated water 20 (illustrated in FIG. 1A). As illustrated in FIG. 2A. in certain embodiments, the ozone generator 242 can be positioned substantially within the water reservoir 240. Alternatively, the ozone generator 242 can be located in another suitable position, such as being positioned substantially adjacent to the water reservoir 240.

It is appreciated that in the embodiment in which the ozone dispenser 214 is fixedly attached to the cleaning device 12, the ozone generator 242 would be permanently built-in and/or integrated within the ozone dispenser 214, such as substantially within and/or adjacent to the water reservoir 240.

The ozone generator 242 can have any suitable design for purposes of generating ozone in the source water 16 in order to convert the source water 16 to the desired ozonated water 20. In many embodiments, the ozone generator 242 can be an electrolytic cell-type ozone generator that includes an electrolytic cell 242A (illustrated in phantom) that can be positioned directly within a volume of the source water 16. In such embodiment, the electrolytic cell 242A is configured to break down at least a portion of the source water 16 into its component parts, i.e. hydrogen and oxygen, and then converts at least a portion of the liberated oxygen into ozone, with the ozone then being directly dissolved into the source water 16 to provide the desired ozonated water 20.

More specifically, in an electrolytic cell-type ozone generator, the electrolytic cell 242A uses electricity at two electrodes, an anode and a cathode, to split water molecules and form gases at the electrodes. The electricity provided to the electrolytic cell 242A and/or the individual electrodes, can be provided via the power source 244. In particular, selective activation of the power source 244 can send the necessary electricity to the electrolytic cell 242A and/or in the individual electrodes to split the molecules of the source water 16 and form the gases at the electrodes.

At the anode, electrons are removed from the water, protons are released into the water and oxygen typically forms on the anode. If certain electrode materials are used at the anode, then it is possible to make ozone as well as diatomic oxygen at the anode. For example, electrode materials such as glassy carbons including boron-doped diamond, platinum, oxides of lead and tin, and other materials may be used to electrolytically produce ozone. In another part of the electrolytic process, hydrogen is formed at the cathode by recombining the protons in the water and electrons that show up from the supply of electricity to the electrolytic cell.

With the ozone thus being generated directly in the source water 16 through use of the ozone generator 242, the ozone can then be mixed with the source water 16 to generate the desired ozonated water 20.

During use of the electrolytic cell-type ozone generator, it is appreciated that certain parameters of the source water 16 can impact the ability to generate ozone. For example, water temperature and water chemistry can impact the ability to generate ozone in the manner desired. In particular, it is appreciated that cooler water typically enables ozone to be generated more effectively and efficiently.

Alternatively, the ozone generator 242 can have another suitable design. For example, in one non-exclusive alternative embodiment, the ozone generator 242 can be a corona discharge-type ozone generator. It is appreciated, however, that different and/or additional components would typically be required for using a different type of ozone generator, such as a corona discharge-type ozone generator. Still alternatively, the ozone generator 242 can have still another suitable design.

As described, it is appreciated that with the electrolytic cell-type ozone generator, the electrolytic cell 242A creates the ozone directly in the source water 16 without any introduction of ambient air. In comparison, the corona discharge-type ozone generator pulls in ambient air and converts the oxygen in that air into ozone gas, which would then be introduced into the source water 16 via an ozone incorporator, such as a venturi device. This results in larger bubbles of ozone gas with the corona discharge-type ozone generator that are then semi-dissolved in the source water 16. In contrast, with the electrolytic cell-type ozone generator, the ozone created by the electrolytic cell 242A creates microbubbles or nanobubbles of ozone gas directly in the source water 16 that, thus, results in higher rates of dissolved ozone gas and considerably less ozone off gassing.

The power source 244 can have any suitable design for purposes of effectively providing necessary power to the ozone generator 242 during use of the ozone dispenser 214. For example, in certain non-exclusive embodiments, the power source 244 can include one or more batteries, which can be rechargeable. Alternatively, the power source 244 can have another suitable design.

The dispenser nozzle 246 is the mechanism formed into the ozone dispenser 214 through which the ozonated water 20 can be dispensed and/or sprayed as desired for performing desired cleaning procedures. In some embodiments, the dispenser nozzle 246 is configured such that the ozonated water 20 is dispensed and/or sprayed in aerosol form. It is appreciated that the dispenser nozzle 246 can be located at any suitable position within the ozone dispenser 214, which may be different than the particular location for the dispenser nozzle 246 shown in FIG. 2A.

The spray activator 248 is configured to selectively activate the ozone dispenser 214 so that the ozonated water 20 is sprayed outwardly away from the ozone dispenser 214. The spray activator 248 can have any suitable design for effectively activating the ozone dispenser 214 to spray the ozonated water 20 in a desired manner. For example, in some non-exclusive embodiments, the spray activator 248 can be an electronic component, such as an electronic trigger or push button, that sends an electronic signal to the ozone dispenser 214 so that the ozonated water 20 is sprayed in the desired manner. In other embodiments, the spray activator 248 can have a manual design that requires an appropriate mechanical linkage assembly between the spray activator 248 and the dispenser nozzle 246 so that the ozonated water 20 is sprayed in the desired manner.

As so described, when the ozone dispenser 214 is being utilized as a stand-alone cleaning mechanism or cleaning agent, i.e. when the ozone dispenser 214 is not attached to the cleaning device 12, the spray activator 248 can be the mechanism that activates the ozone dispenser 214 so that the ozonated water 20 is dispensed via the dispenser nozzle 246.

However, when the ozone dispenser 214 is positioned within the dispenser receiver 28 of the cleaning device 12, the ozone dispenser 214 can have an activator linking mechanism that enables the user to manipulate the dispenser activator 30, which can be coupled to the device handle 26 (illustrated in FIG. 1A) in certain embodiments, in order to selectively dispense the ozonated water 20 via the dispenser nozzle 246.

FIG. 2B is a simplified schematic side view illustration of the ozone dispenser 214 illustrated in FIG. 2A. FIG. 2B provides another viewpoint for seeing the overall size, shape and design of this particular embodiment of the ozone dispenser 214. It is appreciated that FIG. 2B also illustrates the ozone generator 242 (illustrated in phantom), the power source 244 (illustrated in phantom), and the spray activator 248, which can included as part of the ozone dispenser 214.

FIG. 2C is a simplified schematic top view illustration of the ozone dispenser 214 illustrated in FIG. 2A.

FIG. 3 is a simplified schematic perspective view illustration of another embodiment of the cleaner assembly 310. The cleaner assembly 310 shown in FIG. 3 is somewhat similar to the previous embodiments. For example, as illustrated, the cleaner assembly 310 again includes a cleaning device 312 in the form of a mop, including a device body 322, a device head 324, and a device handle 326; and an ozone dispenser 314 that is attached to the cleaning device 312 and/or the device body 322. FIG. 3 further illustrates that, in certain embodiments, the cleaner assembly 310 can again include a dispenser activator 330 that can be coupled to the device handle 326, and which is usable to selectively activate the ozone dispenser 314. It is noted that the device body 322, the device head 324, and the device handle 326 of the cleaning device 312 are substantially identical to the embodiment illustrated and described in detail in relation to FIG. 1A. Accordingly, details of such components of the cleaning device 312 will not be repeated.

However, in this embodiment, the ozone dispenser 314 is fixedly attached directly to the device body 322 of the cleaning device 312. With such design, there is no need for a dispenser receiver such as illustrated in the previous embodiments.

It is further noted that the design and operation of the ozone dispenser 314 and its components, such as the water reservoir 340, the ozone generator 342, the power source 344 (illustrated in phantom), the dispenser nozzle 346, and the spray activator (not clearly visible in FIG. 3), are substantially similar to those illustrated and described in detail herein above, although the precise size and positioning if the ozone dispenser 314 and its components may vary.

FIG. 4 is a simplified schematic perspective view illustration of still another embodiment of the cleaner assembly 410. The cleaner assembly 410 shown in FIG. 4 is somewhat similar to the previous embodiments. More particularly, as illustrated, FIG. 4 again shows that the cleaner assembly 410 includes a cleaning device 412, and an ozone dispenser 414 that is attached to the cleaning device 412. However, in this embodiment, the cleaning device 412 is provided in the form of a vacuum cleaner, such as a wet-dry vacuum cleaner.

As shown, similar to the previous embodiments, although the cleaning device 412 is a vacuum cleaner rather than a mop, the cleaning device 412 again includes a device body 422, a device head 424 that is coupled to the device body 422 near a first body end 422A of the device body 422, a device handle 426 that is coupled to the device body 422 near a second body end 422B of the device body 422, a dispenser receiver 428, and a dispenser activator 430. Moreover, as illustrated the device head 424 again includes a floor-facing surface 424A that is configured to face in a generally downward direction, such as toward the surface 32 (illustrated in FIG. 1A), during use of the cleaner assembly 410. As noted above, in this embodiment, the cleaning device 412 can further include a suction assembly 450 and a collection receptacle 452 that enable the cleaning device 412 to function as a vacuum cleaner in a desired manner.

Similar to the previous embodiments, in some embodiments, the ozone dispenser 414 can be fixedly attached to the cleaning device 412, while in other embodiments, the ozone dispenser 414 can be removably attached to the cleaning device 412, such as through the selective use of the dispenser receiver 428. As above, in embodiments in which the ozone dispenser 414 is removably attached to the cleaning device 412, the ozone dispenser 414 can be selectively removed from the dispenser receiver 428 to be used as a stand-alone cleaning agent or cleaning mechanism, independent of its use as part of the cleaner assembly 410.

In many embodiments, the device body 422 can again be generally cylindrical pole-shaped, and can extend between the device head 424 and the device handle 426. The device body 422 can further have any suitable length, with different lengths for the device body 422 being made available for users of different heights.

The device body 422 can be made from any suitable materials. For example, in certain non-exclusive alternative embodiments, the device body 422 can be made from plastic, fiberglass, aluminum, or steel. Alternatively, the device body 422 can be made from other suitable materials.

The device head 424 can be coupled to the first body end 422A of the device body 422 in any suitable manner. In some embodiments, the device head 424 can be movably coupled to the first body end 422A of the device body 422. For example, in certain embodiments, the device head 424 can be pivotably and/or rotatably coupled to the first body end 422A of the device body 422 to enable the device head 424 to pivot and/or rotate relative to the device body 422. With such design, it is much easier to maintain the floor-facing surface 424A of the device head 424 in engagement with the surface 32, even as the orientation of the device body 422 relative to the surface 32 changes.

The device head 424 is configured to engage the surface 32 in order to effectively remove dirt, dust, debris, etc. from the surface 32. More particularly, operation of the suction assembly 450 enables the cleaning device to suction such dirt, dust, debris, etc. from the surface 32 to be moved into the collection receptacle 452.

The device head 424 can be made from any suitable materials. For example, in certain non-exclusive alternative embodiments, the device head 424 can be made from plastic, fiberglass, aluminum, or steel. Alternatively, the device head 424 can be made from other suitable materials.

The device handle 426 can be coupled to the second body end 422B of the device body 422 in any suitable manner. In some embodiments, the device handle 426 can be fixedly coupled to the second body end 422B of the device body 422.

The device handle 426 is intended to be grasped and held by the user during use of the cleaner assembly 410. It is appreciated that the device handle 426 can have any suitable design for purposes of enabling the user to comfortably grasp and hold the device handle 426 during use of the device assembly 410. For example, in some embodiments, the device handle 426 can have an ergonomic design that enables improved comfort for the user of the cleaning device 412 and/or the cleaner assembly 410. In some embodiments, the device handle 426 can be padded to further assist in the comfort for the user.

The dispenser activator 430 can be substantially similar in design and function to the previous embodiments. More specifically, the dispenser activator 430 can again be configured to selectively activate the ozone dispenser 414 so that the ozonated water 20 (illustrated in FIG. 1A) is sprayed onto the surface 32 near the device head 424. In many embodiments, such as shown in FIG. 4, the dispenser activator 430 can be coupled to the device handle 426. Alternatively, the dispenser activator 430 can be positioned in another suitable manner, such as at any suitable position along a length of the device body 422.

The dispenser activator 430 can again have any suitable design for effectively activating the ozone dispenser 414 to spray the ozonated water 20 onto the surface 32 near the device head 424, such as in the form of an electronic trigger or a manual trigger, or with a push-button design. Alternatively, the dispenser activator 430 can have another suitable design.

Additionally, as above, in alternative embodiments, the ozone dispenser 414 can effectively spray or dispense the ozonated water 20 onto the surface 32 through the ozone dispenser 414 itself; or the ozone dispenser 414 and/or the cleaner assembly 410 as a whole, can be configured to effectively spray or dispense the ozonated water 20 from the ozone dispenser 414, such as into and through a dispenser coordinator (not shown) and/or the device head 424, and onto the surface 32.

It is further noted that the design and operation of the ozone dispenser 414 and its components, such as the water reservoir, the ozone generator, the power source, the dispenser nozzle, and the spray activator, can be substantially similar to those illustrated and described in detail herein above, although the precise size and positioning if the ozone dispenser 414 and its components may vary.

It is understood that although a number of different embodiments of the cleaner assembly 10 and/or the ozone dispenser 14 have been illustrated and described herein, one or more features of any one embodiment can be combined with one or more features of one or more of the other embodiments, provided that such combination satisfies the intent of the present invention.

While a number of exemplary aspects and embodiments of the cleaner assembly 10 and/or the ozone dispenser 14 have been discussed herein above, those of skill in the art will recognize certain modifications, permutations, additions and sub-combinations thereof. It is therefore intended that the present invention shall be interpreted to include all such modifications, permutations, additions and sub-combinations as are within their true spirit and scope, and no limitations are intended to the details of construction or design herein shown.

1. A cleaner assembly for cleaning a floor, the cleaner assembly comprising:
a cleaning device; and
an ozone dispenser that is attachable to the cleaning device.

2. The cleaner assembly of claim 1 wherein the ozone dispenser is removably attachable to the cleaning device.

3. The cleaner assembly of claim 1 wherein the ozone dispenser is fixedly attached to the cleaning device.

4. The cleaner assembly of claim 1 wherein the cleaning device includes (i) a device body having a first body end and an opposed second body end, (ii) a device head that is coupled to the first body end, and (iii) a device handle that is coupled to the second body end.

5. The cleaner assembly of claim 4 wherein the device head includes a substantially planar floor-facing surface that is configured to face the floor during a cleaning procedure.

6. The cleaner assembly of claim 5 wherein the cleaner assembly further includes at least one cleaning pad that is removably coupled to the floor-facing surface.

7. The cleaner assembly of claim 4 wherein the cleaning device further includes a dispenser receiver to is configured to receive and retain the ozone dispenser.

8. The cleaner assembly of claim 4 wherein the cleaning device further includes a dispenser activator that is configured to selectively activate the ozone dispenser, the dispenser activator including an electronic mechanism that is electronically linked to the ozone dispenser to selectively activate the ozone dispenser.

9. The cleaner assembly of claim 4 wherein the cleaning device further includes a dispenser activator that is configured to selectively activate the ozone dispenser, the dispenser activator including a manual mechanism that is mechanically linked to the ozone dispenser to selectively activate the ozone dispenser.

10. The cleaner assembly of claim 1 wherein the ozone dispenser includes a water reservoir that is configured to receive and retain a volume of source water from a water source; and an ozone generator that is positioned one of substantially within and substantially adjacent to the water reservoir, the ozone generator being configured to generate ozone within the source water retained within the water reservoir to generate ozonated water.

11. The cleaner assembly of claim 10 wherein the ozone generator is an electrolytic ozone generator that includes an electrolytic cell that is positioned within the water reservoir.

12. The cleaner assembly of claim 10 wherein the ozone generator is a corona discharge ozone generator.

13. The cleaner assembly of claim 10 wherein the ozone dispenser further includes a spray activator that is configured to selectively activate the ozone dispenser when the ozone dispenser is not attached to the cleaning device.

14. The cleaner assembly of claim 1 wherein the cleaning device is a mop.

15. The cleaner assembly of claim 1 wherein the cleaning device is a vacuum cleaner.

16. A cleaner assembly for cleaning a floor, the cleaner assembly comprising:
a cleaning device including (i) a device body having a first body end and an opposed second body end; (ii) a device head that is movably coupled to the first body end, the device head includes a substantially planar floor-facing surface that is configured to face the floor during a cleaning procedure; (iii) a device handle that is coupled to the second body end; and (iv) a dispenser activator that is configured to selectively activate the ozone dispenser, the dispenser activator including one of an electronic mechanism that is electronically linked to the ozone dispenser to selectively activate the ozone dispenser and a manual mechanism that is mechanically linked to the ozone dispenser to selectively activate the ozone dispenser; and
an ozone dispenser that is attachable to the cleaning device, the ozone dispenser including (i) a water reservoir that is configured to receive and retain a volume of source water from a water source; (ii) an ozone generator that is positioned one of substantially within and substantially adjacent to the water reservoir, the ozone generator being configured to generate ozone within the source water retained within the water reservoir to generate ozonated water; (iii) a power source that is configured to provide power to the ozone generator; and (iv) a dispenser nozzle through which the ozonated water is dispensed from within the water reservoir.

17. The cleaner assembly of claim 16 wherein the ozone dispenser is removably attachable to the cleaning device.

18. The cleaner assembly of claim 16 wherein the ozone dispenser is fixedly attached to the cleaning device.

19. The cleaner assembly of claim 16 wherein the cleaning device is a mop.

20. The cleaner assembly of claim 16 wherein the cleaning device is a vacuum cleaner.
